(19) United States
(12) Patent Application Publication
(10) Pub. No.: US 2021/0380645 A1
(43) Pub. Date: Dec. 9, 2021

(54) Efficient Protein Expression System for Expressing Bt Insecticidal Protein

(71) Applicants: SYNGENTA PARTICIPATIONS AG, Basel (CH); SYNGENTA BIOTECHNOLOGY CHINA CO., LTD., Beijing (CN)

(72) Inventor: Qian Zheng, Beijing (CN)

(73) Assignee: SYNGENTA PARTICIPATIONS AG, Basel (CH)

(21) Appl. No.: 17/286,125

(22) PCT Filed: Oct. 14, 2019
(86) PCT No.: PCT/CN2019/111040

§ 371 Date: Apr. 16, 2021

(30) Foreign Application Priority Data
Oct. 18, 2018 (CN) 201811213835.X

(51) Int. Cl. C07K 14/245; C12N 9/86; C12N 15/70

(52) U.S. Cl. CPC C07K 14/245; C12Y 305/02006; C12N 15/70; C12N 9/86

(57) ABSTRACT

An efficient protein expression system for expressing a Bt insecticidal protein. Said protein expression system uses Bla (beta-lactamase) as a fusion tag, so that Bt genes unable to be expressed as soluble proteins are expressed as soluble proteins.

### TECHNICAL FIELD

The present invention relates to the field of protein (in particular, Bt protein) expression. The present invention provides an efficient protein expression system for expressing a Bt insecticidal protein. The protein expression system uses Bla (beta-lactamase) as a fusion tag, so that Bt genes unable to be expressed as soluble proteins are expressed efficiently as soluble proteins.

### BACKGROUND ART

In vitro expression technology of a protein is an important part of genetic engineering technology. Compared with other protein expression systems, Escherichia coli is an efficient and economical way, and the expression level of a recombinant protein can reach 50% of the total protein content of Escherichia coli. The recombinant protein with a normal biochemical activity is usually in a soluble form. Therefore, in order to obtain active proteins, soluble expression pathways are usually used.

A Bt protein is a parasporal crystal protein of Bacillus thuringiensis formed in bacteria during a sporulation period. The Bt protein is a widely used biopesticide, which is safe and has a high-efficiency and broad-spectrum insecticidal activity, and is harmless to humans, animals and non-target pests. Exploring new Bt proteins has very high commercial application value.

Expresso® Solubility & Expression Screening System from Lucigen is a protein expression tag screening kit containing 7 fusion tags. Gene fragments can be conveniently connected to 8 different vectors through homologous recombination reactions. Through the rapid screening of 7 different fusion tags, the most suitable tag for expressing the target protein is obtained (see Table 1 below).

TABLE 1

| # | Fusion tag | AA length | kDa* | pI | Description |
|---|---|---|---|---|---|
| 1 | 6xHis-AFV | 113 | 13.5 | 5.0 | Hypothetical protein from |
| | | | | | Acidianus filovirus 1 |
| 2 | 6xHis-SlyD | 210 | 22.7 | 5.2 | FKBP-type peptidylprolyl |
| | | | | | cis-trans isomerase |
| 3 | 6xHis-Tsf | 297 | 32.2 | 5.7 | E. coli elongation factor |
| 4 | 6xHis-SUMO | 115 | 13.3 | 5.2 | Small ubiquitin-like |
| | | | | | modifier |
| 5 | 6xHis-Bla | 381 | 41.3 | 4.4 | Beta-lactamase |
| 6 | 6xHis-MBP | 382 | 42.1 | 5.5 | Maltose-binding protein |
| 7 | 6xHis-GST | 233 | 27.4 | 6.6 | Glutathione S-transferase |
| 8 | 6xHis control | 14 | 1.8 | 7.0 | Affinity tag |

The newly explored Bt genes need to be expressed as soluble proteins to test the insecticidal activity thereof. However, relying on the current Escherichia coli expression technology, only about 50% to 60% of Bt genes can be successfully expressed. Therefore, the successful expression of Bt genes as soluble proteins has important research and commercial value.

### SUMMARY OF THE INVENTION

The present invention establishes an efficient protein expression system. Using the existing Escherichia coli expression technology, about 50% to 60% of Bt genes can be successfully expressed as soluble proteins. Using the method of the present invention, the remaining 40% to 50% of Bt genes unable to be expressed as soluble proteins can achieve 80% successful expression (expressed as soluble proteins), and the total success rate of Bt gene expression can reach at least 80%.

In the present invention, 16 Bt genes unable to be expressed as soluble proteins were initially used as test samples and screened for fusion tags using 8 vectors in Expresso® Solubility & Expression Screening System from Lucigen. The results showed that the pSol-Bla vector could achieve 100% expression for 16 test samples. For the convenience of cloning, we modified the pSol-Bla vector from Lucigen and used the modified vector to express an additional 237 Bt genes, which are unable to be expressed as soluble proteins, in Escherichia coli; as a result, 193 genes were successfully expressed as soluble proteins, and the expression success rate was 81.4%. Therefore, for a total of 253 (16+237) Bt genes unable to be expressed as soluble proteins before, 209 (16+193) genes were successfully expressed as soluble proteins, and the expression success rate was 82.6%.

In one aspect, the present invention relates to the use of Bla, i.e., beta-lactamase, as a fusion tag for expressing a Bt protein.

In another aspect, the present invention relates to the use of a modified pSol-Bla vector as shown in FIG. 1 for expressing a Bt protein.

Preferably, in the above-mentioned uses, the Bt protein is expressed in Escherichia coli. More preferably, the Escherichia coli is Escherichia coli 10G or BL21 (DE3). Preferably, in the above-mentioned uses, the Bt protein is a Bt protein unable to be expressed.

In another aspect, the present invention relates to a fusion protein comprising a Bt protein and a beta-lactamase as a fusion tag. Preferably, the Bt protein is a Bt protein unable to be expressed.

In another aspect, the present invention relates to a nucleic acid molecule encoding the above-mentioned fusion protein.

In another aspect, the present invention relates to a vector comprising the above-mentioned nucleic acid molecule. Preferably, the vector is a pSol-Bla vector into which a Bt gene to be expressed is inserted, or a modified pSol-Bla vector as shown in FIG. 1 into which a Bt gene to be expressed is inserted.

In another aspect, the present invention relates to a cell comprising the above-mentioned vector. Preferably, the cell is an Escherichia coli cell. More preferably, the Escherichia coli cell is an Escherichia coli 10G or BL21 (DE3) cell.

In another aspect, the present invention relates to a method for expressing a Bt protein, comprising: (1) culturing the above-mentioned cell under conditions suitable for expressing the desired protein; and (2) optionally, cleaving the Bla tag to obtain a Bt protein without the tag.

In another aspect, the present invention relates to a modified pSol-Bla vector as shown in FIG. 1.

### BRIEF DESCRIPTION OF THE DRAWINGS

FIG. 1 is a map showing the modified pSol-Bla vector of the present invention.

### DETAILED DESCRIPTION OF EMBODIMENTS

The embodiments of the present invention will be described in detail below in combination with examples, but a person skilled in the art would understand that the following examples serve only to illustrate the present invention and are not intended to limit the scope of the present invention. If the specific conditions are not indicated in the examples, the conventional conditions or the conditions suggested by the manufacturer shall be followed. Any reagents or instruments used, unless the manufacture stated, are conventional products that can be obtained by market purchase.

### Example 1. Screening of Fusion Tags for 16 Bt Genes Unable to be Expressed as Soluble Proteins to Achieve Soluble Expression

We selected 16 Bt genes unable to be expressed as soluble proteins through conventional methods (for example, the expression of pET series vectors at different temperatures and IPTG induced concentrations, the use of fusion tags such as SUMO and GST, the use of different types of competent cells, cell-free culture, expression with Bt cells, etc.). Their numbers, molecular weights, and expression issues are shown in Table 2 below:

TABLE 2

| Protein | | | Molecular | | Expression |
|---|---|---|---|---|---|
| number | Gene family | SEQ ID NO | weight | | issue |
| BT-0025 | Cry1If | 1 | 80 | KD | Insoluble |
| BT-0027 | | 2 | 79 | KD | No expression |
| BT-0275 | Cry22Aa | 3 | 96 | KD | No expression |
| BT-0362 | | 4 | 73 | KD | No expression |
| BT-0425 | Cry36Aa | 5 | 56 | KD | No expression |
| BT-0435 | Cry42Aa | 6 | 75 | KD | No expression |
| BT-0445 | Vip1Aa | 7 | 138 | KD | Insoluble |
| BT-0452 | Cry19Ca | 8 | 59 | KD | No expression |
| BT-0560 | Cry20Aa | 9 | 26 | KD | No expression |
| BT-0587 | Cry49Ab | 10 | 60 | KD | No expression |
| BT-0669 | Cry2Ae | 11 | 30 | KD | Insoluble |
| BT-0685 | Cry49Ab | 12 | 51 | KD | Insoluble |
| BT-0827 | Cry48Ab2 | 13 | 86 | KD | No expression |
| BT-0830 | Cry48Ab2 | 14 | 87 | KD | No expression |
| BT-0831 | Cry54Ba1 | 15 | 80 | KD | No expression |
| HO4 | Cry1Ab.1Ca | 16 | 130 | KD | Insoluble |

Using Expresso® Solubility and Expression Screening System from Lucigen, according to the instructions thereof, the primer sequences at both ends were designed to synthesize PCR primers of these 16 genes, respectively. The target fragments of 16 genes with homologous recombination ends were obtained by PCR amplification methods. Then, after mixing the PCR products with 8 linear vectors in the kit, the chemically competent cells were transformed with plasmids. After the positive clones were identified from the obtained monoclonal plasmids by PCR, the positive clones were sent to a company for sequencing and identification. After analyzing the sequencing results, we obtained the plasmids of these 16 genes on 8 vectors, a total of 128 plasmids.

30 μl of Escherichia coli 10G or BL21 (DE3) glycerol bacteria with these 16 genes were cultured in 3 ml of LB medium at 37° C. to an OD600 of approximately 0.5, and then 20% rhamnose was added to induce culture overnight at 18° C. The bacteria were collected and subjected to disruption by sonication, and whole bacteria and supernatant were collected respectively and boiled for 10 minutes before the expression was analyzed by SDS-PAGE. The expression of these 128 plasmids in Escherichia coli 10G and BL21 (DE3) cells was shown in Table 3 below (the first 6 genes in Table 3 were purified after successful expression and the Bla tag was cleaved to obtain a pure soluble target protein).

TABLE 3

| | Issue when | | |
|---|---|---|---|
| | expressing | | |
| | through | | |
| | conventional | Valid tag | |
| Protein | methods | 10 G | BL21 (DE3) |
| BT-0025 (80 KD) | Insoluble | 6 tags (Bla, GST, | 6 tags (Bla, GST, |
| | | His, MBP, SUMO, Tsf) | His, MBP, SUMO, Tsf) |
| BT-0827 (86 KD) | No | 4 tags (AFV | Not tested |
| | expression | Bla, GST, Tsf) | |
| BT-0830 (87 KD) | No | 5 tags (Bla, GST, | Not tested |
| | expression | MBP, Tsf, SlyD) | |
| BT-0831 (80 KD) | No | 6 tags (AFV, Bla, | Not tested |
| | expression | GST, MBP, SlyD, Tsf) | |
| HO4 (130 KD) | Insoluble | 4 tags (AFV, | Not tested |
| | | Bla, MBP, Tsf) | |
| BT-0027 (79 KD) | No | 3 tags (Bla, | Not tested |
| | expression | MBP, SlyD) | |
| BT-0685 (51 KD) | Insoluble | All 8 tags | 5 tags (AFV, Bla, |
| | | | GST, MBP, His) |
| BT-0669 (30 KD) | Insoluble | 7 tags (All, | 7 tags (All |
| | | except for GST) | except for GST) |
| BT-0587 (60 KD) | No | 6 tags (AFV, Bla, | All 8 tags |
| | expression | GST, His, MBP, SUMO) | |
| BT-0560 (26 KD) | No | 3 tags (Bla, MBT, Tsf) | 4 tags (AFV, |
| | expression | | GST, MBP, SUMO) |
| BT-0362 (73 KD) | No | 4 tags (Bla, MBP, | Not tested |
| | expression | SlyD, Tsf) | |
| BT-0425 (56 KD) | No | 5 tags (AFV, Bla, | Not tested |
| | expression | MBP, SUMO, Tsf) | |
| BT-0452 (59 KD) | No | 5 tags (AFV, Bla, | Not tested |
| | expression | GST, MBP, SlyD) | |
| BT-0435 (75 KD) | No | 2 tags (AFV, Bla) | Not tested |
| | expression | | |
| BT-0445 (138 KD) | Insoluble | 4 tags (AFV, | 4 tags (AFV, Bla, |
| | | Bla, MBP, Tsf) | MBP, Tsf) |
| BT-0275 (96 KD) | No | 2 tags (AFV, Bla) | Not tested |
| | expression | | |

The results showed that most of the 8 vectors with different tags in the kit could express individual genes in these 16 Bt genes, and only the pSol-Bla vector was effective for all 16 genes (that is, the genes could be expressed as soluble proteins), implying that the Bla tag specifically improved the effect of the Bt gene expression.

### Example 2. Modification of pSol-Bla Vector from Lucigen and Use of the Modified pSol-Bla Vector on 237 Bt Genes Unable to be Expressed as Soluble Proteins

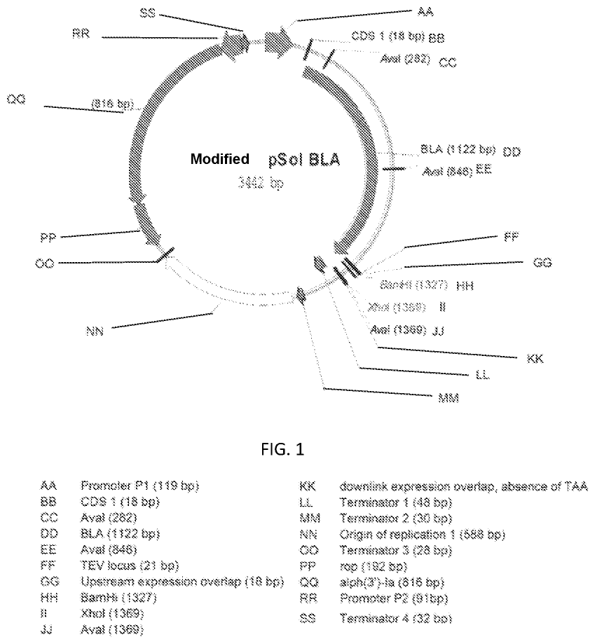

For the convenience of cloning, we made a few modifications to the pSol-Bla vector from Lucigen, wherein the stop codon TAA at positions 1326-1328 in the original vector was removed, and the sequence “ggatccggctcaggctcaggctcaggctcaggctcaggctcactcgag” was added at position 1326 of the original vector, the sequence comprising one BamHI enzyme digestion site and one XhoI enzyme digestion site. The modified pSol-Bla vector was as shown in FIG. 1. We selected 237 Bt genes unable to be expressed as soluble proteins (using a pET28a vector, the soluble form of protein cannot be obtained in the supernatant of the cell lysate under conditions of induction overnight with 0.2-0.5 mM IPTG at 18° C.), and cloned the genes into the modified pSol-Bla vector through enzyme digestion and ligation. After transforming the vector into Escherichia coli 10G cells, we used the same method to express these 237 plasmids: 30 μl of glycerol bacteria were added to 3 ml of LB medium and cultured at 37° C. to an OD600 of approximately 0.5, and then 20% rhamnose was added to induce the culture overnight at 18° C. The bacteria were collected and subjected to disruption by sonication, and whole bacteria and supernatant were collected respectively and boiled for 10 minutes before the expression was analyzed by SDS-PAGE.

The results showed that 193 out of the 237 Bt genes unable to be expressed as soluble proteins obtained soluble fusion proteins in this way, and the success rate was up to 81.4%. This result showed that the vector fused with Bla could specifically increase the success rate of the successful expression of the Bt gene.

The information regarding the 237 Bt genes unable to be expressed as soluble proteins is as follows:

TABLE 4

| | | | Molecular | |
|---|---|---|---|---|
| Protein | | | weight | Whether expressed |
| number | Gene family | SEQ ID NO | (KD) | in this experiment |
| BT-0269 | Cry41Ab | 17 | 93 | Yes |
| BT-0290 | Cry19Ca | 18 | 136 | No |
| BT-0397 | Cry59Aa | 19 | 77 | Yes |
| BT-0398 | Cry4Ca | 20 | 75 | Yes |
| BT-0411 | Vip2Ad | 21 | 22.5 | Yes |
| BT-0426 | Cry59Aa | 22 | 73 | Yes |
| BT-0434 | Cry21Ba | 23 | 120 | Yes |
| BT-0439 | Cry36Aa | 24 | 57 | Yes |
| BT-0441 | Cry68Aa | 25 | 82 | Yes |
| BT-0455 | Cry41Ba | 26 | 94 | Yes |
| BT-0456 | Cry22Ab | 27 | 79 | Yes |
| BT-0464 | Cry44Aa | 28 | 59 | Yes |
| BT-0474 | Cry41Ba | 29 | 92 | Yes |
| BT-0481 | Cry24Ca | 30 | 45 | Yes |
| BT-0482 | Cry4Cc | 31 | 49 | Yes |
| BT-0483 | Cry4Ca | 32 | 131 | Yes |
| BT-0526 | Cry2Aa | 33 | 39 | Yes |
| BT-0532 | Vip2Af | 34 | 50 | Yes |
| BT-0552 | Cry15Aa | 35 | 34 | Yes |
| BT-0555 | Cry52Aa | 36 | 77 | Yes |
| BT-0557 | Cry53Ab | 37 | 74 | No |
| BT-0567 | Cry70Bb | 38 | 106 | Yes |
| BT-0579 | Cry40Da | 39 | 74 | Yes |
| BT-0581 | Cry40Da | 40 | 72 | Yes |
| BT-0585 | Cry36Aa | 41 | 52 | Yes |
| BT-0586 | Cry49Aa | 42 | 56 | Yes |
| BT-0591 | Cry4Cc | 43 | 78 | No |
| BT-0596 | Cry4Ca | 44 | 61 | Yes |
| BT-0597 | Cry69Aa | 45 | 135 | No |
| BT-0599 | Cry54Aa | 46 | 72 | Yes |
| BT-0600 | Cry56Aa | 47 | 70 | Yes |
| BT-0603 | Cry53Ab | 48 | 76 | Yes |
| BT-0609 | Cry4-like | 49 | 26 | Yes |
| BT-0610 | Cry68Aa | 50 | 89 | Yes |
| BT-0612 | Cry15Aa | 51 | 40 | No |
| BT-0613 | Cry51Aa | 52 | 37 | Yes |
| BT-0624 | Cry22Ab | 53 | 64 | Yes |
| BT-0626 | Cry49Aa | 54 | 23 | Yes |
| BT-0663 | Cry68Aa | 55 | 82 | Yes |
| BT-0666 | Cry68Aa | 56 | 90 | Yes |
| BT-0667 | Cry68Aa | 57 | 91 | Yes |
| BT-0671 | Cry14Ab | 58 | 90 | Yes |
| BT-0681 | Cry42Aa | 59 | 88 | Yes |
| BT-0683 | Cry19Ca | 60 | 76 | Yes |
| BT-0713 | Cry41Aa1 | 61 | 56 | Yes |
| BT-0735 | Cry4Cb1 | 62 | 79 | Yes |
| BT-0744 | Cry60Aa | 63 | 35 | Yes |
| BT-0760 | Cry64Aa1 | 64 | 37 | No |
| BT-0762 | Cry64Aa1 | 65 | 36 | No |
| BT-0764 | Cry15Aa1 | 66 | 34 | Yes |
| BT-0766 | Cry54Aa2 | 67 | 78 | Yes |
| BT-0767 | Cry20Ba1 | 68 | 83 | Yes |
| BT-0768 | Cry51Aa1 | 69 | 35 | No |
| BT-0770 | Cry51Aa1 | 70 | 40 | Yes |
| BT-0772 | Cry70Bb1 | 71 | 110 | No |
| BT-0776 | Cry32Mb1 | 72 | 138 | No |
| BT-0777 | Cry32Ra1 | 73 | 126 | Yes |
| BT-0778 | Cry32Cb1 | 74 | 96 | Yes |
| BT-0781 | cry69Aa1 | 75 | 81 | Yes |
| BT-0783 | Cry40Da1 | 76 | 74 | Yes |
| BT-0784 | Cry4Ca1 | 77 | 65 | No |
| BT-0785 | Cry55Aa1 | 78 | 35 | Yes |
| BT-0787 | Cry55Aa1 | 79 | 35 | No |
| BT-0788 | Cry60Aa1 | 80 | 36 | Yes |
| BT-0795 | Cry41Aa1 | 81 | 66 | Yes |
| BT-0878 | Cry4Ca1 | | 66 | Yes |
| BT-0879 | Cry40Da1 | | 74 | Yes |
| BT-0895 | Cry49Ab1 | | 43 | Yes |
| BT-0903 | Vip2Ba2 | | 44 | Yes |
| BT-0905 | Vip2Ac1 | | 58 | Yes |
| BT-0911 | Cry14Aa1 | 82 | 91 | Yes |
| BT-0912 | Cry32Ta1 | 83 | 147 | Yes |
| BT-0914 | Cry73Aa1 | 84 | 89 | Yes |
| BT-0915 | Cry32Sa1 | 85 | 139 | Yes |
| BT-0916 | Cry41Ba2 | 86 | 86 | Yes |
| BT-0920 | Cry21Ca1 | 87 | 146 | Yes |
| BT-0922 | Cry4Aa4 | 88 | 137 | Yes |
| BT-0927 | Cry8Ga3 | 89 | 37 | Yes |
| BT-0934 | Cyt2Ca1 | 90 | 25 | Yes |
| BT-0936 | Cry49Ab1 | 91 | 31 | Yes |
| BT-0938 | Cry49Ab1 | 92 | 42 | Yes |
| BT-0939 | Cry13Aa1 | 93 | 68 | Yes |
| BT-0941 | Cry32Ra1 | 94 | 155 | No |
| BT-0942 | Cry60Aa3 | 95 | 36 | Yes |
| BT-0943 | Cry8La1 | 96 | 115 | Yes |
| BT-0944 | Cry30Db1 | 97 | 78 | Yes |
| BT-0946 | Cry51Aa2 | 98 | 32 | Yes |
| BT-0948 | Cry32Da1 | 99 | 138 | Yes |
| BT-0949 | Cry8Ca1 | 100 | 132 | Yes |
| BT-0950 | Cry60Ba3 | 101 | 36 | Yes |
| BT-0951 | Cry32Ra1 | 102 | 147 | Yes |
| BT-0952 | Cry73Aa1 | 103 | 59 | Yes |
| BT-0953 | Cry73Aa1 | 104 | 57 | Yes |
| BT-0954 | Cry45Aa | 105 | 31 | Yes |
| BT-0955 | Cry32Qa1 | 106 | 150 | Yes |
| BT-0956 | Cry20Aa1 | 107 | 81 | Yes |
| BT-0957 | Cry54Aa2 | 108 | 77 | Yes |
| BT-0958 | Cry60Aa3 | 109 | 36 | Yes |
| BT-0961 | Cry41Ba2 | 110 | 63 | No |
| BT-0962 | Cry19Ca1 | 111 | 86 | Yes |
| BT-0964 | Cry28Aa2 | 112 | 125 | Yes |
| BT-0965 | Cry60Ba3 | 113 | 35 | Yes |
| BT-0968 | Cry43Ca1 | 114 | 145 | Yes |
| BT-0970 | Cry4Ca1 | 115 | 139 | Yes |
| BT-0976 | Cry8Ib1 | 116 | 137 | No |
| BT-0977 | Cry22Ba1 | 117 | 53 | Yes |
| BT-0979 | Cry42Aa1 | 118 | 57 | Yes |
| BT-0980 | Cry30Aa1 | 119 | 78 | Yes |
| BT-0981 | Cry4Ca1 | 120 | 136 | Yes |
| BT-0983 | Cry54Aa2 | 121 | 80 | Yes |
| BT-0984 | Cry4Cb2 | 122 | 129 | Yes |
| BT-0985 | Cyt2Ca1 | 123 | 29 | Yes |
| BT-0987 | Cry36Aa1 | 124 | 54 | Yes |
| BT-0989 | Cry4Ca1 | 125 | 64 | Yes |
| BT-0991 | Cry40Da1 | 126 | 76 | Yes |
| BT-0993 | Cry70Bb1 | 127 | 113 | Yes |
| BT-0997 | Cry8Ia1 | 128 | 139 | No |
| BT-0998 | Cry45Aa | 129 | 29 | Yes |
| BT-0999 | Cry45Aa | 130 | 30 | Yes |
| BT-1000 | Cry32Ra1 | 131 | 151 | Yes |
| BT-1002 | Cry11Bb1 | 132 | 33 | Yes |
| BT-1003 | Cry56Aa2 | 133 | 78 | Yes |
| BT-1004 | Cry53Ab1 | 134 | 76 | Yes |
| BT-1006 | Cry60Ba3 | 135 | 35 | Yes |
| BT-1008 | Cry60Ba3 | 136 | 35 | Yes |
| BT-1009 | Cry10Aa4 | 137 | 82 | No |
| BT-1010 | Cry9Ga1 | 138 | 84 | Yes |
| BT-1013 | Cry32Ca1 | 139 | 145 | Yes |
| BT-1015 | Cry32Qa1 | 140 | 143 | No |
| BT-1018 | Vip1Ba2 | 141 | 88 | Yes |
| BT-1019 | Cry60Ba3 | 142 | 32 | Yes |
| BT-1020 | Cry2Ah1 | 143 | 57 | Yes |
| BT-1021 | Cry70Bb1 | 144 | 109 | Yes |
| BT-1022 | Cry30Ga2 | 145 | 81 | No |
| BT-1023 | Cry39Aa1 | 146 | 78 | Yes |
| BT-1026 | Cry56Aa2 | 147 | 73 | Yes |
| BT-1027 | Cry72Aa1 | 148 | 79 | Yes |
| BT-1028 | Cry30Fa1 | 149 | 79 | Yes |
| BT-1029 | Cyt2Aa4 | | 29 | No |
| BT-1031 | Cry40Da1 | 150 | 86 | Yes |
| BT-1032 | Cry4Ca1 | 151 | 64 | Yes |
| BT-1033 | Cry73Aa1 | 152 | 64 | Yes |
| BT-1034 | Vip2Ae2 | 153 | 84 | Yes |
| BT-1036 | Cry32Qa1 | 154 | 143 | Yes |
| BT-1037 | Cry54Ba1 | 155 | 34 | Yes |
| BT-1042 | Cry4Ba5 | 156 | 134 | Yes |
| BT-1045 | Cry67Aa2 | 157 | 125 | Yes |
| BT-1048 | Cry32Cb1 | 158 | 144 | Yes |
| BT-1050 | Vip3Ad1 | 159 | 104 | Yes |
| BT-1051 | Cry73Aa1 | 160 | 54 | Yes |
| BT-1054 | Cry2Aa3 | 161 | 36 | Yes |
| BT-1055 | Cry73Aa1 | 162 | 58 | Yes |
| BT-1057 | Cry15Aa1 | 163 | 38 | Yes |
| BT-1060 | Cry4Ba5 | 164 | 140 | No |
| BT-1062 | Cry8Aa1 | 165 | 77 | Yes |
| BT-1064 | Cry8Ga3 | 166 | 129 | Yes |
| BT-1065 | Cry29Aa1 | 167 | 78 | Yes |
| BT-1068 | Cry51Aa2 | 168 | 32 | Yes |
| BT-1069 | Cry70Bb1 | 169 | 93 | Yes |
| BT-1070 | Cry4Aa4 | 170 | 134 | No |
| BT-1073 | Cry32La1 | 171 | 137 | No |
| BT-1076 | Cry60Aa3 | 172 | 40 | No |
| BT-1078 | Cry22Aa3 | 173 | 33 | Yes |
| BT-1084 | Cry33Aa1 | 174 | 30 | Yes |
| BT-1085 | Cry32La1 | 175 | 128 | Yes |
| BT-1087 | Cry32La1 | 176 | 75 | Yes |
| BT-1088 | Cry73Aa1 | 177 | 91 | Yes |
| BT-1090 | Cyt2Aa4 | 178 | 31 | Yes |
| BT-1091 | Cry32Ra1 | 179 | 151 | No |
| BT-1092 | Cry51Aa1 | 180 | 35 | Yes |
| BT-1093 | Vip1Aa2 | 181 | 88 | Yes |
| BT-1094 | Cry73Aa1 | 182 | 94 | Yes |
| BT-1095 | Cry32Ra1 | 183 | 147 | Yes |
| BT-1102 | Vip3Ca2 | 184 | 105 | Yes |
| BT-1106 | Cry42Aa1 | 185 | 55 | Yes |
| BT-1110 | Cry6Aa3 | 186 | 42 | No |
| BT-1113 | Cry32Ra1 | 187 | 150 | Yes |
| BT-1114 | Cry41Aa1 | 188 | 96 | Yes |
| BT-1116 | Cry15Aa1 | 189 | 37 | Yes |
| BT-1120 | Cry41Ba2 | 190 | 109 | Yes |
| BT-1121 | Cry43Ca1 | 191 | 137 | Yes |
| BT-1122 | Cry64Aa1 | 192 | 33 | Yes |
| BT-1124 | CryL (BT-0174) | 193 | 119 | Yes |
| BT-1125 | Cry73Aa1 | 194 | 55 | Yes |
| BT-1126 | Cry16Aa1 | 195 | 50 | Yes |
| BT-1127 | Cry29Aa1 | 196 | 61 | Yes |
| BT-1272 | Cry66Aa2 | 197 | 159 | Yes |
| BT-1274 | Vip1Ba1 | 198 | 97 | Yes |
| BT-1275 | Cry55Aa1 | 199 | 44 | Yes |
| BT-1276 | Cry55Aa1 | 200 | 40 | Yes |
| BT-1277 | Cry1Fb4 | 201 | 53 | Yes |
| BT-1280 | Cry1Ia11 | 202 | 83 | Yes |
| BT-1283 | Cry12Aa1 | 203 | 96 | Yes |
| BT-1284 | Cry15Aa1 | 204 | 40 | Yes |
| BT-1285 | Cry60Ba3 | 205 | 35 | No |
| BT-1287 | Cry60Aa1 | 206 | 35 | No |
| BT-1292 | Cry71Aa1 | 207 | 71 | Yes |
| BT-1296 | Cry22Ab2 | 208 | 93 | Yes |
| BT-1297 | Cry22Ba1 | 209 | 99 | No |
| BT-1300 | Cry32Ra1 | 210 | 149 | No |
| BT-1305 | Cry37Aa1 | 211 | 16 | Yes |
| BT-1306 | Cry4Ca1 | 212 | 60 | Yes |
| BT-1308 | Cry59Aa1 | 213 | 75 | No |
| BT-1312 | Cry45Aa | 214 | 31 | Yes |
| BT-1314 | Cry36Aa1 | 215 | 57 | Yes |
| BT-1316 | Cry49Ab1 | 216 | 43 | No |
| BT-1317 | Cry36Aa1 | 217 | 55 | Yes |
| BT-1318 | Cry49Ab1 | 218 | 44 | No |
| BT-1319 | Cry49Ab1 | 219 | 43 | Yes |
| BT-1322 | Cry49Aa4 | 220 | 29 | Yes |
| BT-1323 | Cry4Aa1 | 221 | 80 | No |
| BT-1324 | Cry4Ba5 | 222 | 79 | Yes |
| BT-1331 | Cyt2Aa3 | 223 | 27 | Yes |
| BT-1333 | Cry73Aa1 | 224 | 57 | Yes |
| BT-1334 | Cry73Aa1 | 225 | 57 | Yes |
| BT-1336 | Cry64Aa1 | 226 | 40 | Yes |
| BT-1337 | Cry45Aa | 227 | 41 | Yes |
| BT-1338 | Cry45Aa | 228 | 48 | Yes |
| BT-1339 | Cry60Aa1 | 229 | 38 | Yes |
| BT-1340 | Vip1Bb1 | 230 | 108 | Yes |
| BT-1342 | Vip2Ba1 | 231 | 29 | No |
| BT-1343 | Vip2Ba2 | 232 | 29 | No |
| BT-1344 | Vip2Ac1 | 233 | 28 | No |
| BT-1345 | Vip2Ba2 | 234 | 30 | Yes |
| BT-1346 | Vip2Aa1 | 235 | 33 | No |
| BT-1347 | Vip2Ba1 | 236 | 30 | Yes |
| BT-1353 | Vip2Ba2 | 237 | 29 | No |
| BT-1354 | Vip2Ac1 | 238 | 26 | No |
| BT-1356 | Vip2Aa2 | 239 | 25 | No |
| BT-1357 | Vip2Ba2 | 240 | 29 | No |
| BT-1358 | Vip2Aa2 | 241 | 26 | No |
| BT-1359 | Vip2Aa1 | 242 | 28 | Yes |
| BT-1360 | Vip2Ad1 | 243 | 57 | No |
| BT-1362 | Vip1Bb1 | 244 | 118 | Yes |
| BT-1366 | Cry22Aa3 | 245 | 75 | Yes |
| BT-1369 | Cry3Ca1 | 246 | 67 | Yes |
| BT-1371 | Cry22Aa3 | 247 | 122 | Yes |

### REFERENCES

1. Tokunaga Hi, et al., (2010) Appl Microbiol Biotechnol. 88, 1223
2. Bruce E. Tabashnik, et al., Sci Rep. 2015; 5: 15107

1-5. (canceled)

6. A fusion protein comprising a Bt protein and a beta-lactamase as a fusion tag.

7. The fusion protein according to claim 6, wherein the Bt protein is a Bt protein unable to be expressed.

8. A nucleic acid molecule encoding the fusion protein according to claim 6.

9. A vector comprising the nucleic acid molecule according to claim 8.

10. The vector according to claim 9, wherein the vector is a pSol-Bla vector into which a Bt gene to be expressed is inserted, or a modified pSol-Bla vector as shown in FIG. 1 into which a Bt gene to be expressed is inserted.

11. A cell comprising the vector according to claim 9.

12. The cell according to claim 11, wherein the cell is an Escherichia coli cell.

13. The cell according to claim 12, wherein the Escherichia coli cell is an Escherichia coli 10G or BL21 (DE3) cell.

14. A method for expressing a Bt protein, comprising:
(1) culturing the cell according to claim 11 under conditions suitable for expressing the desired protein; and
(2) optionally, cleaving the Bla tag to obtain a Bt protein without the tag.

15. A modified pSol-Bla vector as shown in FIG. 1.
